(19) United States
(12) Patent Application Publication
(10) Pub. No.: US 2026/0137319 A1
(43) Pub. Date: May 21, 2026

(54) REAL-TIME ATTENTION DEFICIT HYPERACTIVITY DISORDER SCREENING SYSTEM USING GRAPHICS PROCESSING UNIT ACCELERATED ELECTROENCEPHALOGRAM ANALYSIS

(71) Applicants: Mahesh KHADATARE, San Jose, CA (US); Pragati DHARMALE, San Jose, CA (US)

(72) Inventors: Mahesh KHADATARE, San Jose, CA (US); Pragati DHARMALE, San Jose, CA (US)

(21) Appl. No.: 19/441,619

(22) Filed: Jan. 6, 2026

(51) Int. Cl. A61B 5/16; A61B 5/00; A61B 5/291; A61B 5/31; A61B 5/372

(52) U.S. Cl. CPC A61B 5/168; A61B 5/291; A61B 5/31; A61B 5/372; A61B 5/721; A61B 5/7267

(57) ABSTRACT

The present invention relates to a system and method for real-time screening of Attention Deficit Hyperactivity Disorder using electroencephalographic signals processed through a GPU-accelerated time-frequency inference architecture. The invention enables continuous acquisition of multi-channel electroencephalographic data, adaptive preprocessing for artifact suppression and signal stabilization, and parallel execution of time-frequency transformations to extract neurologically relevant features in real time. Extracted features are analyzed using an inference process configured to identify neurological patterns associated with Attention Deficit Hyperactivity Disorder, while continuous validation of signal quality and temporal consistency ensures diagnostic reliability. The system dynamically adapts analytical parameters based on evolving signal characteristics and regulates computational workload to achieve energy-efficient operation during prolonged monitoring. The invention provides a technically integrated, machine-implemented screening solution capable of delivering reliable, real-time neurological assessment suitable for clinical environments.

### TECHNICAL FIELD OF THE INVENTION

The present invention relates to the field of neurodiagnostic computing systems and medical signal processing, and more particularly to a machine-implemented system and method for real-time screening of Attention Deficit Hyperactivity Disorder using electroencephalographic signals processed through GPU-accelerated time-frequency inference architectures. The invention specifically pertains to computational devices and structural systems configured to acquire, process, validate, and interpret EEG signals using high-throughput parallel processing to enable clinically reliable and real-time neurological screening.

### BACKGROUND OF THE INVENTION

Electroencephalography-based neurological assessment has long been used to study brain activity; however, conventional ADHD screening methods relying on behavioral assessment, questionnaire-based scoring, or offline EEG analysis suffer from subjectivity, delayed interpretation, and limited temporal resolution. Existing EEG-based diagnostic systems typically rely on CPU-bound processing pipelines that are incapable of performing high-resolution time-frequency transformations in real time, thereby restricting their clinical applicability during live assessment scenarios. Furthermore, traditional EEG analysis systems are constrained by fixed windowing techniques, static frequency bands, and non-adaptive thresholds, which result in reduced sensitivity to transient neurological signatures associated with ADHD.

Current clinical EEG systems also lack integrated computational validation mechanisms capable of correlating multi-dimensional brainwave features with ADHD-specific neurological markers under continuous monitoring conditions. The absence of GPU-accelerated inference engines prevents real-time spectral decomposition and dynamic feature extraction, leading to delayed diagnostic outputs and reduced reliability. Additionally, prior art systems do not adequately integrate machine learning-driven inference with adaptive time-frequency analysis, nor do they provide structural device configurations capable of seamless deployment within clinical environments. These limitations collectively necessitate a dedicated machine-based system that overcomes computational latency, enhances diagnostic fidelity, and enables continuous, real-time ADHD screening.

Electroencephalography-based neurological assessment has long been explored as a potential objective aid for identifying cognitive and neurodevelopmental conditions such as Attention Deficit Hyperactivity Disorder. ADHD is characterized by complex neurophysiological patterns involving alterations in attention regulation, impulse control, and executive functioning, which manifest as subtle variations in brainwave activity across multiple frequency bands. Traditional clinical diagnosis of ADHD has primarily relied on behavioral observation, psychological questionnaires, and clinician judgment, which, although widely accepted, are inherently subjective and susceptible to inter-observer variability, cultural bias, and inconsistent reporting. As a result, there has been sustained interest in developing EEG-based systems capable of providing quantitative neurological markers to support or augment clinical diagnosis.

Early EEG-based ADHD assessment systems typically focused on offline signal acquisition followed by post hoc spectral analysis. These systems relied on fixed-band frequency analysis, such as evaluating theta-to-beta ratios computed over long EEG recording sessions. While such approaches demonstrated some correlation with ADHD-related neurophysiological traits, they suffered from limited temporal resolution and an inability to capture transient or context-dependent brainwave dynamics. Moreover, offline processing introduced delays between data acquisition and diagnostic interpretation, rendering such systems unsuitable for real-time screening or continuous monitoring. The static nature of these analyses also failed to account for intra-subject variability, changes in cognitive state, or artifacts introduced during EEG recording.

With advancements in digital signal processing, more sophisticated EEG analysis techniques were introduced, including short-time Fourier transforms, wavelet-based methods, and time-frequency representations. Although these techniques improved temporal localization and frequency resolution, their implementation in practical clinical systems remained constrained by computational limitations. Most existing solutions employed central processing unit-based architectures that processed EEG channels sequentially or in limited parallel fashion. As EEG systems expanded to include higher channel counts and higher sampling rates, CPU-based processing pipelines struggled to meet real-time constraints, leading to compromises in resolution, simplified feature extraction, or reduced channel utilization.

Another limitation of existing EEG-based ADHD screening solutions lies in their reliance on predefined, rigid frequency windows and manually tuned thresholds. Such systems assume that ADHD-related neurological signatures remain stable across individuals and contexts, which is inconsistent with contemporary neuroscience findings. ADHD manifests heterogeneously, with significant inter-individual differences influenced by age, cognitive load, comorbid conditions, and environmental factors. Static models are therefore prone to misclassification, either by failing to detect atypical patterns or by falsely identifying non-ADHD neurological variations as pathological. Existing systems rarely incorporate adaptive mechanisms capable of recalibrating frequency bands, thresholds, or feature relevance based on ongoing data.

Several commercial and research-grade EEG solutions have attempted to incorporate machine learning techniques to improve diagnostic performance. These systems typically employ classifiers trained on extracted EEG features to differentiate ADHD from non-ADHD patterns. However, in many implementations, machine learning is applied after extensive preprocessing and dimensionality reduction, often performed offline or using simplified real-time approximations. This separation between signal processing and inference introduces bottlenecks and limits the ability of the system to adapt dynamically to evolving EEG patterns. Furthermore, many such solutions depend on pre-trained models that are not updated during deployment, reducing robustness when applied to populations or recording conditions different from the training dataset.

A further drawback of existing solutions is the lack of integrated computational validation mechanisms. EEG signals are highly susceptible to noise, motion artifacts, electrode impedance fluctuations, and environmental interference. Many prior systems perform artifact removal using static filters or heuristic rules without continuously validating signal integrity during analysis. As a result, corrupted or low-quality signals may still propagate through the processing pipeline, leading to unreliable diagnostic outputs. In clinical settings, this undermines trust in automated screening systems and necessitates manual review by specialists, negating the intended efficiency gains.

Energy efficiency and scalability also present significant challenges in current EEG-based ADHD screening technologies. High-resolution time-frequency analysis and multi-channel processing demand substantial computational resources, particularly when attempting real-time operation. CPU-centric systems often consume excessive power or require high-performance workstations, limiting portability and increasing operational costs. Edge-deployed or bedside screening solutions are therefore constrained by hardware limitations, making continuous or widespread screening impractical in resource-limited environments.

Another important limitation of existing approaches is their poor integration with clinical workflows. Many EEG analysis systems function as standalone research tools requiring specialized operators and post-processing expertise. The lack of seamless integration with electronic medical records, clinical decision support systems, and real-time visualization interfaces restricts their adoption in routine clinical practice. Moreover, existing systems rarely provide transparent reasoning or confidence measures alongside screening outputs, which further reduces clinician acceptance in high-stakes diagnostic contexts such as ADHD evaluation.

The absence of dedicated GPU-accelerated architectures in most prior solutions represents a critical technological gap. Modern graphical processing units are inherently suited for parallel computation tasks such as time-frequency transformation, matrix operations, and neural network inference. Despite this, many existing EEG screening systems either underutilize GPU resources or rely on generic acceleration without tailoring techniques specifically for neurological signal characteristics. Consequently, they fail to achieve the full potential of real-time, high-resolution EEG analysis necessary for reliable ADHD screening.

Additionally, prior art systems often lack structural device configurations optimized for continuous neurological monitoring. EEG acquisition hardware, processing units, and inference components are frequently treated as loosely coupled subsystems rather than as an integrated machine. This fragmented architecture introduces data transfer latency, synchronization issues, and reduced fault tolerance. In contrast, a structurally unified machine capable of tightly coupling EEG acquisition with GPU-based processing and inference is essential for maintaining deterministic performance and diagnostic reliability in real-time screening scenarios.

In summary, existing solutions for EEG-based ADHD screening are limited by subjective diagnostic reliance, offline or delayed processing, rigid analytical models, insufficient computational power, lack of adaptive inference, inadequate signal validation, high energy consumption, and poor clinical integration. These drawbacks collectively hinder the practical deployment of EEG-based ADHD screening as a reliable, real-time diagnostic support tool. There remains a clear and unmet need for a technically advanced system that integrates GPU-accelerated time-frequency analysis, adaptive inference, continuous validation, and machine-level structural integration to overcome the limitations of prior art and enable robust, real-time ADHD screening suitable for modern clinical environments.

### OBJECTS OF THE INVENTION

An object of the present invention is to provide a real-time ADHD screening system that utilizes GPU-accelerated computation to perform high-resolution time-frequency analysis of EEG signals without interrupting live signal acquisition. Another object is to provide a machine-implemented device structure capable of integrating EEG acquisition hardware, GPU processing units, adaptive inference logic, and clinical output interfaces within a single operational architecture. A further object is to enable adaptive neurological screening through self-optimizing frequency inference and computational validation mechanisms that improve diagnostic accuracy over time. Yet another object is to ensure clinical deployability through energy-efficient computation, fault-tolerant processing, and secure data handling within hospital and diagnostic environments.

### SUMMARY OF THE INVENTION

The present invention discloses a system and method for real-time ADHD screening using EEG signals processed through a GPU-accelerated time-frequency inference framework. The system comprises a dedicated machine structure integrating EEG signal acquisition hardware, a parallel processing unit configured as a graphical processing unit, a time-frequency transformation processor, and a neurological inference unit trained to identify ADHD-associated spectral patterns. The GPU architecture enables concurrent processing of multiple EEG channels, allowing dynamic windowing, adaptive spectral resolution, and real-time feature extraction without latency.

The system further incorporates computational validation logic that continuously verifies signal consistency, frequency stability, and temporal coherence to reduce false positives and enhance screening reliability. Machine learning-based inference mechanisms correlate extracted EEG features with ADHD-specific neurological signatures, producing screening outputs that are dynamically refined through adaptive learning. The overall architecture is designed for continuous operation, enabling sustained monitoring and immediate diagnostic feedback while maintaining compliance with clinical workflow requirements.

An object of the present invention is to provide a technically advanced system and method for real-time screening of Attention Deficit Hyperactivity Disorder using electroencephalographic signals, wherein neurological assessment is performed through continuous time-frequency analysis without reliance on subjective behavioral evaluation alone, thereby improving objectivity, consistency, and clinical reliability of ADHD screening.

Another object of the invention is to overcome the computational limitations of conventional EEG-based screening systems by employing a GPU-accelerated processing architecture capable of executing high-resolution, multi-channel time-frequency transformations in real time, thereby eliminating latency associated with offline analysis and enabling immediate neurological inference during live EEG acquisition.

A further object of the invention is to enable adaptive neurological characterization by dynamically adjusting frequency segmentation, temporal windowing, and inference thresholds based on incoming EEG signal properties, subject-specific neurological variability, and contextual assessment conditions, thereby reducing false positives and improving sensitivity to diverse ADHD-related brainwave patterns.

Another object of the invention is to integrate machine learning-based inference directly within the real-time EEG processing pipeline such that extracted spectral features are immediately evaluated against learned neurological patterns associated with ADHD, allowing continuous refinement of screening accuracy through adaptive learning while maintaining deterministic performance during clinical operation.

An additional object of the invention is to incorporate continuous computational validation mechanisms that assess signal integrity, noise levels, temporal coherence, and frequency stability throughout the screening process, ensuring that diagnostic outputs are derived only from neurologically reliable EEG data and reducing the risk of misclassification due to artifacts or degraded signal quality.

Yet another object of the invention is to provide a structurally integrated machine or device configuration that tightly couples EEG acquisition hardware, GPU-based processing units, inference logic, memory subsystems, and clinical output interfaces within a single operational architecture, thereby minimizing data transfer delays, improving fault tolerance, and ensuring consistent real-time performance.

An object of the invention is also to achieve energy-efficient operation by intelligently managing computational workloads across GPU resources, enabling sustained real-time screening during prolonged monitoring sessions without excessive power consumption, thus supporting deployment in clinical, portable, and point-of-care environments.

Another object of the invention is to facilitate seamless integration with clinical workflows by generating real-time screening outputs, confidence indicators, and neurological trends in a format compatible with diagnostic review and medical record systems, thereby enhancing clinician usability and adoption without disrupting established clinical practices.

A further object of the invention is to enable scalable deployment across different EEG configurations, patient populations, and clinical settings by supporting variable channel counts, adaptable processing parameters, and extensible inference models, ensuring robustness and applicability across diverse neurological assessment scenarios.

An object of the invention is to provide a reliable and repeatable real-time ADHD screening framework that supports early identification, continuous monitoring, and decision support while maintaining data integrity, patient confidentiality, and clinical compliance, thereby addressing the limitations of existing EEG-based diagnostic systems and advancing the state of neurodiagnostic technology.

### BRIEF DESCRIPTION OF FIGURES

These and other features, aspects, and advantages of the present invention will become better understood when the following detailed description is read concerning the accompanying drawings in which like characters represent like parts throughout the drawings, wherein:

FIG. 1 displays a block diagram of a system for real-time screening of Attention Deficit Hyperactivity Disorder using electroencephalographic signals; and

FIG. 2 displays flow chart of a method for real-time screening of Attention Deficit Hyperactivity Disorder using electroencephalographic signals

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION OF THE INVENTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

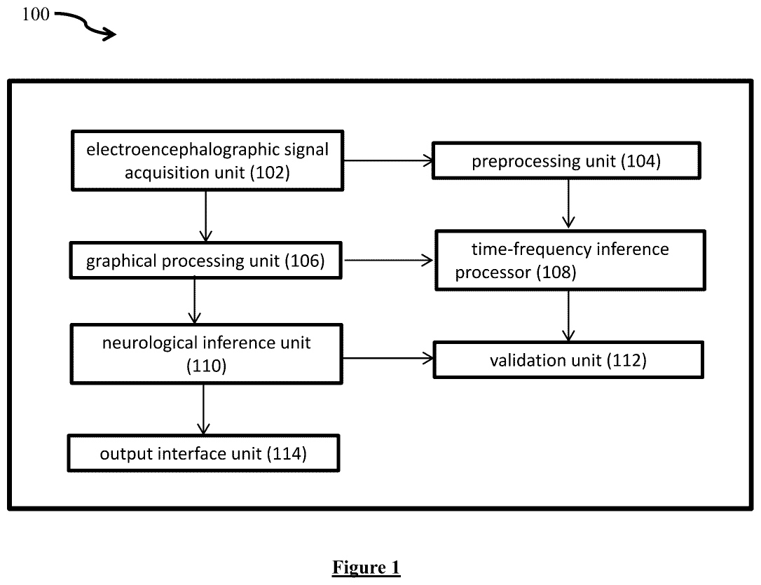

Referring to FIG. 1, a block diagram of a system for real-time screening of Attention Deficit Hyperactivity Disorder using electroencephalographic signals is illustrated. The system 100 comprises: an electroencephalographic signal acquisition unit (102)configured to receive analog brainwave signals from a plurality of scalp-mounted electrodes and convert the received signals into digitized signal streams; a preprocessing unit (104)operatively coupled to the electroencephalographic signal acquisition unit and configured to condition the digitized signal streams by performing baseline stabilization, artifact attenuation, and temporal alignment across multiple channels; a graphical processing unit (106) operatively coupled to the preprocessing unit, the graphical processing unit being configured as a parallel computation processor to execute concurrent time-frequency transformations on the conditioned signal streams in real time; a time-frequency inference processor (108) implemented within the graphical processing unit and configured to dynamically segment the conditioned signal streams into adaptive temporal windows and frequency partitions based on detected signal characteristics; a neurological inference unit (110) operatively coupled to the graphical processing unit and configured to analyze extracted time-frequency features to determine neurological patterns associated with Attention Deficit Hyperactivity Disorder; a validation unit (112) configured to continuously assess signal integrity, frequency stability, and temporal consistency of the processed signals prior to generation of screening outputs; and an output interface unit (114) configured to generate real-time screening indicators indicative of a likelihood of Attention Deficit Hyperactivity Disorder for clinical review. In an embodiment, the electroencephalographic signal acquisition unit (102) comprises a plurality of amplification circuits, analog-to-digital conversion circuits, and synchronization circuits configured to maintain channel-level temporal coherence across all digitized signal streams prior to preprocessing.

In an embodiment, the preprocessing unit (104) is configured to detect and suppress motion-induced artifacts, electrode impedance fluctuations, and environmental noise components by applying adaptive filtering parameters derived from real-time signal variance measurements. In an embodiment, the graphical processing unit (106) is configured to execute multiple time-frequency transformations concurrently across the plurality of electroencephalographic channels by distributing computation threads such that latency between signal acquisition and feature extraction remains within a predetermined real-time threshold.

In an embodiment, the time-frequency inference processor (108) is configured to vary temporal window lengths and frequency resolution in response to detected changes in signal non-stationarity, cognitive state transitions, or abrupt spectral deviations associated with neurological activity.

In an embodiment, the neurological inference unit (110) comprises a trained computational model stored in a memory unit, the computational model being configured to correlate extracted time-frequency features with stored neurological reference patterns indicative of Attention Deficit Hyperactivity Disorder.

In an embodiment, the neurological inference unit (110) is further configured to update internal weighting parameters of the computational model based on accumulated screening outcomes and validated signal patterns, thereby enabling adaptive improvement of screening accuracy over successive screening sessions.

In an embodiment, the validation unit (112) is configured to inhibit generation of screening outputs when one or more signal quality parameters fall outside predefined neurological reliability limits, thereby preventing screening decisions based on degraded or unreliable electroencephalographic data.

In an embodiment, further comprising a memory unit operatively coupled to the preprocessing unit, graphical processing unit, and neurological inference unit, the memory unit being configured to store raw electroencephalographic data, processed time-frequency features, validation metrics, and screening results for subsequent clinical analysis.

In an embodiment, the output interface unit (114) is configured to provide continuous visualization of neurological trends, screening confidence indicators, and temporal progression of inferred attention-related patterns through a clinician-accessible display interface. Each of the components recited in the system claims is implemented as a physical, hardware-based element formed of electronic circuitry and embedded processing resources, and not as a mere abstract or software construct. The electroencephalographic signal acquisition unit is realized as a tangible electronic assembly comprising physical scalp-mounted electrodes, conductive signal paths, analog front-end circuitry including amplifiers and filters, and on-board analog-to-digital conversion hardware that produces digitized signal streams from sensed brainwave voltages. The preprocessing unit is implemented as a dedicated hardware processing block comprising digital signal conditioning circuitry, clocked logic elements, and embedded processors configured to execute baseline stabilization, artifact attenuation, and temporal alignment through hardware-level arithmetic and control operations. The graphical processing unit is a physical parallel computation processor fabricated on a semiconductor substrate and includes multiple processing cores, on-chip memory blocks, and hardware thread schedulers that execute concurrent time-frequency transformations directly on the conditioned signal streams. The time-frequency inference processor is realized as a hardware-implemented processing logic instantiated within the graphical processing unit and operates through dedicated computational circuits and memory access pathways to perform dynamic temporal windowing and frequency partitioning. The neurological inference unit is implemented as a hardware processing unit comprising one or more physical processors and associated memory circuitry that store and execute trained computational parameters to analyze extracted time-frequency features. The validation unit is a hardware-based monitoring circuit comprising comparators, counters, and control logic that continuously evaluates signal integrity, frequency stability, and temporal consistency prior to permitting output generation. The output interface unit is a physical hardware interface comprising display driving circuitry, signal output ports, and control electronics configured to generate and present real-time screening indicators, while the memory unit is a tangible data storage device formed of non-transitory memory hardware electrically coupled to the other units for storing raw signals, processed features, validation metrics, and screening results.

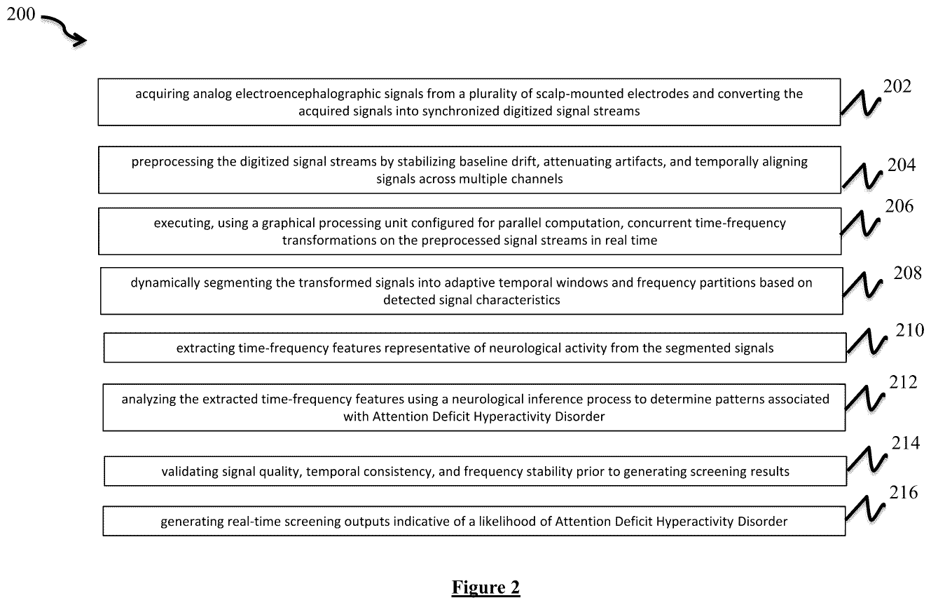

Referring to FIG. 2, a flow chart for a method for real-time screening of Attention Deficit Hyperactivity Disorder using electroencephalographic signals is illustrated. The method 200 is capable of being implemented by system illustrated in FIG. 1. The method 200 comprises:
At step 202, the method 200 includes acquiring analog electroencephalographic signals from a plurality of scalp-mounted electrodes and converting the acquired signals into synchronized digitized signal streams;
At step 204, the method 200 includes preprocessing the digitized signal streams by stabilizing baseline drift, attenuating artifacts, and temporally aligning signals across multiple channels;
At step 206, the method 200 includes executing, using a graphical processing unit configured for parallel computation, concurrent time-frequency transformations on the preprocessed signal streams in real time;
At step 208, the method 200 includes dynamically segmenting the transformed signals into adaptive temporal windows and frequency partitions based on detected signal characteristics;
At step 210, the method 200 includes extracting time-frequency features representative of neurological activity from the segmented signals;
At step 212, the method 200 includes analyzing the extracted time-frequency features using a neurological inference process to determine patterns associated with Attention Deficit Hyperactivity Disorder;
At step 214, the method 200 includes validating signal quality, temporal consistency, and frequency stability prior to generating screening results; and
At step 216, the method 200 includes generating real-time screening outputs indicative of a likelihood of Attention Deficit Hyperactivity Disorder.

In an embodiment, acquiring the electroencephalographic signals comprises amplifying low-amplitude brainwave signals, digitizing the amplified signals using analog-to-digital conversion, and synchronizing the digitized signals across all channels using a common timing reference. In an embodiment, preprocessing the digitized signal streams comprises detecting motion-related disturbances, electrode impedance variations, and environmental interference, and selectively suppressing the detected disturbances using adaptive filtering parameters derived from real-time signal variance measurements.

In an embodiment, executing concurrent time-frequency transformations comprises distributing signal processing tasks across multiple parallel computation threads within the graphical processing unit such that multiple electroencephalographic channels are processed simultaneously without introducing acquisition-to-inference latency.

In an embodiment, dynamically segmenting the transformed signals comprises adjusting temporal window lengths in response to detected non-stationary signal behavior and modifying frequency resolution based on observed spectral transitions associated with neurological activity.

In an embodiment, extracting the time-frequency features comprises identifying frequency band energy distributions, temporal power variations, and cross-band relationships indicative of attention-related neurological patterns.

In an embodiment, analyzing the extracted time-frequency features comprises comparing the extracted features with stored neurological reference patterns using a trained computational model to determine similarity to Attention Deficit Hyperactivity Disorder-associated signatures.

In an embodiment, further comprising updating internal weighting parameters of the trained computational model based on accumulated screening outcomes and validated signal features to improve screening accuracy over successive screening sessions.

In an embodiment, validating signal quality comprises continuously monitoring signal-to-noise characteristics, frequency coherence, and temporal continuity, and inhibiting generation of screening outputs when one or more validation parameters fall outside predefined neurological reliability limits.

In an embodiment, further comprising storing raw electroencephalographic data, processed time-frequency features, validation metrics, and screening results in a memory unit for subsequent clinical review and longitudinal neurological assessment.

In an embodiment, amplifying the low-amplitude brainwave signals comprises applying channel-specific gain control by initially measuring baseline amplitude distributions for each of the plurality of scalp-mounted electrodes during an initialization interval, dynamically adjusting amplification factors for each channel based on the measured distributions to prevent saturation and clipping, and continuously recalibrating the amplification factors during acquisition in response to slow amplitude drift detected in the digitized signal streams, wherein synchronizing the digitized signals across all channels using the common timing reference comprises embedding timing markers into each digitized signal stream at acquisition, detecting timing deviations by comparing marker alignment across channels, computing channel-specific timing offsets at sub-sample resolution, and correcting the offsets by interpolative resampling prior to preprocessing to maintain phase coherence across the plurality of scalp-mounted electrodes.

In this embodiment, the amplification and synchronization of low-amplitude brainwave signals are carried out through a tightly coupled adaptive gain control and precision timing correction process that directly addresses physiological variability and hardware-induced timing errors. During an initial initialization interval, each scalp-mounted electrode acquires raw electroencephalographic activity while the subject is maintained in a neutral baseline condition, such as resting with eyes closed. The digitized amplitudes obtained from each channel are statistically analyzed to derive channel-specific amplitude distributions, including measures such as median signal level, variance, and peak excursions. These distributions are used to determine individualized amplification factors such that channels exhibiting inherently weaker signals due to scalp impedance, electrode placement, or anatomical differences receive higher gain, while channels with stronger signals are constrained to prevent analog front-end saturation and digital clipping. As signal acquisition continues, slow amplitude drift caused by factors such as electrode polarization, skin hydration changes, or gradual movement is detected by monitoring long-term trends in the digitized signal envelopes. The amplification factors are recalibrated incrementally in response to these trends, ensuring that signal resolution is maintained without introducing abrupt gain transitions that could distort neurological features. In parallel, precise temporal synchronization is achieved by embedding high-resolution timing markers, derived from a common timing reference such as a crystal-controlled clock, directly into each channel's digitized signal stream at the point of acquisition. By continuously comparing the alignment of these markers across channels, even minute timing deviations introduced by clock skew, buffer latency, or multiplexing delays are detected. Channel-specific timing offsets are computed at sub-sample resolution and corrected using interpolative resampling techniques prior to preprocessing, effectively realigning the signals in time. This process preserves phase coherence across all electrodes, which is essential for accurately resolving inter-channel phase relationships, functional connectivity, and synchronization patterns. The technical effect achieved by this embodiment is a substantial improvement in signal fidelity and inter-channel consistency, enabling more accurate downstream time-frequency analysis and neurological inference, while the technical advancement lies in the continuous, adaptive correction of both amplitude and timing distortions in real time, which is not achievable with static gain or coarse synchronization approaches.

In an embodiment, detecting motion-related disturbances comprises monitoring abrupt amplitude excursions, transient broadband spectral energy increases, and inter-channel decorrelation events occurring within predefined temporal proximity, classifying detected events as motion-induced disturbances when the events simultaneously affect a minimum subset of spatially adjacent electrodes, and tagging corresponding signal segments for selective suppression during preprocessing; and wherein selectively suppressing the detected disturbances comprises calculating adaptive filtering parameters by estimating real-time signal variance, spectral flatness, and temporal persistence within the tagged signal segments, applying attenuation selectively to affected frequency components while preserving uncorrupted frequency components, and reintegrating the filtered segments into the digitized signal streams without introducing discontinuities between adjacent unfiltered segments.

In this embodiment, motion-related disturbances are identified and mitigated through a multi-dimensional artifact detection and correction process that operates directly on the continuously acquired electroencephalographic signal streams. As acquisition progresses, each channel is monitored for abrupt amplitude excursions that exceed physiologically plausible limits, transient increases in broadband spectral energy that are characteristic of muscle activity or electrode displacement, and sudden drops in inter-channel correlation that indicate non-neuronal perturbations. These indicators are evaluated within tightly bounded temporal intervals so that coincident occurrences across channels can be detected. When such events are observed to simultaneously affect a predefined minimum number of spatially adjacent electrodes, the disturbance is classified as motion-induced rather than neurological in origin, since genuine brain activity typically exhibits more distributed and structured spatial patterns. The corresponding temporal segments of the signal streams are then tagged to indicate the presence of motion artifacts, without immediately discarding the data, thereby preserving contextual continuity for subsequent processing stages.

Selective suppression of the tagged disturbances is performed using adaptive filtering techniques that are derived from real-time signal characteristics within the affected segments. For each tagged segment, the system estimates instantaneous signal variance to quantify amplitude instability, spectral flatness to distinguish structured neural rhythms from noise-like artifacts, and temporal persistence to determine whether the disturbance is impulsive or sustained. These estimates are used to compute filtering parameters that attenuate only the frequency components most strongly associated with the detected artifact, such as high-frequency components linked to muscle activity or low-frequency transients caused by electrode shifts, while leaving intact frequency bands that remain consistent with valid neurological activity. The filtered segments are then reintegrated into the original digitized signal streams using continuity-preserving interpolation and overlap techniques, ensuring smooth transitions at segment boundaries and preventing the introduction of artificial discontinuities or phase distortions. The technical effect achieved by this embodiment is the substantial reduction of motion-induced artifacts without sacrificing valid neurological information, enabling reliable signal analysis even in conditions where subject movement is unavoidable. The technical advancement lies in the context-aware, frequency-selective suppression of artifacts based on spatial and temporal coherence criteria, which enhances signal robustness and screening accuracy compared to conventional blanket filtering or segment rejection approaches.

In an embodiment, detecting electrode impedance variations comprises continuously estimating impedance-related signal distortions by evaluating low-frequency drift magnitude, noise floor elevation, and phase instability for each channel, comparing the estimated distortions against baseline impedance profiles obtained during initialization, and dynamically modifying preprocessing parameters to reduce the influence of channels exhibiting impedance instability on subsequent processing stages, and wherein executing concurrent time-frequency transformations using the graphical processing unit comprises partitioning each preprocessed signal stream into overlapping data blocks, transferring the data blocks into a graphical processing unit memory space using asynchronous data transfer operations, executing parallel transformation operations on the data blocks across multiple processing cores, and assembling transformed outputs in a time-ordered sequence to preserve temporal continuity; and wherein distributing signal processing tasks across multiple parallel computation threads comprises mapping individual electroencephalographic channels to independent thread groups, assigning transformation workloads based on channel-specific signal complexity determined from variance and spectral density measurements, and dynamically balancing thread execution to prevent processing delays in channels exhibiting higher computational demand.

In this embodiment, variations in electrode impedance are detected and managed through continuous estimation of impedance-induced signal distortions that directly affect signal reliability and downstream analytical accuracy. For each electroencephalographic channel, low-frequency drift magnitude is monitored to identify slow baseline wander commonly associated with unstable electrode-skin contact, while elevation of the noise floor is assessed to detect increased thermal or contact noise resulting from impedance changes. In addition, phase instability is evaluated by tracking irregular phase fluctuations over time, which can indicate intermittent contact or micro-movements of the electrode. These distortion indicators are continuously compared against baseline impedance profiles that were established during the initialization interval under stable contact conditions. When deviations beyond acceptable limits are detected, preprocessing parameters are dynamically modified so that channels exhibiting impedance instability contribute less influence to subsequent processing stages. This may include adaptive attenuation, reduced weighting during feature aggregation, or increased smoothing to suppress artifact propagation, thereby preserving overall spatial integrity without discarding entire channels. The technical effect of this approach is enhanced robustness to real-world electrode variability, ensuring that transient contact issues do not disproportionately degrade the quality of the neurological inference.

Concurrently, computational efficiency and real-time performance are achieved by executing time-frequency transformations on a graphical processing unit. Each preprocessed signal stream is partitioned into overlapping data blocks to maintain continuity of temporal features across block boundaries. These blocks are transferred into the graphical processing unit memory using asynchronous data transfer operations that overlap communication with computation, thereby minimizing latency. Parallel transformation operations, such as windowed spectral or time-frequency decompositions, are executed simultaneously across multiple processing cores, and the resulting transformed outputs are assembled in strict time order to preserve temporal coherence across channels. In parallel, signal processing tasks are distributed across multiple computation threads by mapping individual electroencephalographic channels to independent thread groups. Transformation workloads are assigned dynamically based on channel-specific signal complexity, which is determined from real-time variance and spectral density measurements, allowing channels with richer or more volatile signals to receive proportionally greater computational resources. Thread execution is continuously balanced to prevent delays in channels with higher computational demand from stalling the overall pipeline. The technical advancement achieved by this embodiment lies in the combination of adaptive signal quality management with scalable parallel computation, enabling deterministic real-time processing of high-density electroencephalographic data while maintaining high signal fidelity and temporal precision.

In an embodiment, dynamically segmenting the transformed signals into adaptive temporal windows comprises continuously evaluating non-stationary behavior by tracking short-term energy fluctuations and spectral redistribution within the transformed signals, decreasing temporal window duration upon detection of rapid transitions indicative of cognitive state changes, and increasing temporal window duration during periods of sustained spectral stability to capture extended neurological patterns, and wherein dynamically segmenting the transformed signals further comprises recalculating window overlap ratios in response to detected transition frequency, increasing overlap during high-transition intervals to improve temporal resolution and reducing overlap during stable intervals to minimize redundant computation.

In this embodiment, the transformed electroencephalographic signals are segmented into temporal windows whose duration and overlap are continuously adapted in response to the non-stationary nature of brain activity. Rather than relying on fixed-length windows, the system continuously evaluates short-term energy fluctuations and spectral redistribution patterns within the time-frequency transformed signals to assess the rate and intensity of neurological change. Rapid increases in energy gradients, sudden shifts in dominant frequency bands, or redistribution of spectral power across adjacent bands are interpreted as indicators of transient cognitive events such as attentional shifts, stimulus responses, or state transitions. When such rapid transitions are detected, the temporal window duration is automatically reduced so that these short-lived phenomena are captured with higher temporal precision. Conversely, during intervals in which the spectral composition remains stable and energy variations are minimal, the window duration is increased to encompass longer time spans, enabling more robust characterization of sustained neurological patterns such as resting rhythms or prolonged cognitive engagement.

In addition to adjusting window length, the system dynamically recalculates window overlap ratios based on the observed frequency of detected transitions. During high-transition intervals, increased overlap between successive windows is applied to ensure continuity of feature extraction and to avoid loss of temporal detail at window boundaries. This higher overlap allows subtle temporal evolutions to be tracked smoothly across adjacent segments. During periods of sustained stability, overlap is reduced to minimize redundant computation and conserve processing resources without sacrificing informational content. The technical effect of this adaptive segmentation strategy is improved sensitivity to transient neurological events while maintaining efficient processing during stable periods. The technical advancement lies in the real-time, data-driven adjustment of both window duration and overlap, which enables accurate and resource-efficient time-frequency analysis that is better aligned with the inherently dynamic behavior of brain signals than conventional fixed-window approaches.

In an embodiment, decreasing and increasing the temporal window duration further comprises computing a transition intensity score based on combined temporal energy gradient and frequency redistribution rate within each transformed signal segment, mapping the transition intensity score to a bounded temporal window adjustment range, and applying window duration changes gradually across successive segments to prevent abrupt discontinuities in feature extraction, and wherein generating the multi-dimensional feature representations further comprises computing channel-pair interaction descriptors by measuring time-lagged co-activation patterns within each adaptive temporal window, weighting the interaction descriptors based on channel validation confidence derived during signal quality validation, and integrating the weighted interaction descriptors with channel-wise features to form a composite feature set used for neurological inference.

In this embodiment, refinement of temporal window adaptation is achieved by introducing a quantified transition intensity score that governs how temporal window durations are increased or decreased in response to neurological dynamics. For each transformed signal segment, a temporal energy gradient is computed to capture the rate of change in signal power over time, while a frequency redistribution rate is simultaneously calculated to measure how rapidly spectral energy shifts across frequency bands. These two measures are combined to generate a transition intensity score that reflects both temporal and spectral volatility within the segment. The score is then mapped to a predefined and bounded range of allowable window duration adjustments, ensuring that window lengths are modified within safe operational limits that preserve signal integrity. Rather than applying abrupt changes, window duration adjustments are distributed gradually across successive segments, allowing the segmentation process to evolve smoothly over time. This gradual adaptation prevents artificial discontinuities in feature extraction and avoids introducing spurious artifacts that could otherwise arise from sudden changes in temporal resolution. The technical effect of this approach is stable yet responsive temporal segmentation that remains sensitive to genuine neurological transitions while maintaining continuity in the extracted features.

In addition, multi-dimensional feature representations are enhanced by incorporating channel-pair interaction descriptors that capture functional relationships between different scalp regions. Within each adaptive temporal window, time-lagged co-activation patterns between pairs of channels are measured to identify synchronized or sequential neural activity indicative of network-level processes. These interaction descriptors are not treated uniformly; instead, they are weighted according to channel validation confidence scores that are derived during prior signal quality validation stages, reflecting the reliability of each channel at that moment. Channels exhibiting higher signal stability and lower artifact contamination contribute more strongly to the interaction measures, while less reliable channels are proportionally down-weighted. The weighted interaction descriptors are then integrated with channel-wise features, such as band-specific energy or temporal variability, to form a composite feature set. The technical advancement achieved by this embodiment lies in the combination of smoothly adaptive temporal segmentation with reliability-aware network feature construction, resulting in a richer and more robust representation of neurological activity that improves inference accuracy and resilience to signal quality variations.

In an embodiment, dynamically segmenting the transformed signals into frequency partitions comprises monitoring relative power shifts across frequency ranges, reallocating frequency partition boundaries toward ranges exhibiting increased neurological relevance, and maintaining coarser partitions in ranges exhibiting low informational contribution, such that computational emphasis is adaptively concentrated on neurologically active frequency regions, and wherein extracting the time-frequency features comprises generating multi-dimensional feature representations by combining temporally aggregated energy values, inter-frequency coupling indicators, and inter-channel synchrony measurements computed within each adaptive temporal window, and normalizing the feature representations using rolling statistical baselines derived from preceding validated windows.

In this embodiment, the transformed electroencephalographic signals are further adaptively segmented along the frequency dimension to concentrate analytical resolution on neurologically informative regions while reducing unnecessary computation in less relevant bands. Relative power distribution across frequency ranges is continuously monitored within each adaptive temporal window to identify shifts in neurological activity, such as increased power in specific bands associated with cognitive engagement, attention, or pathological markers. When sustained or emerging increases in relative power are detected within particular frequency ranges, the boundaries of the frequency partitions are reallocated to provide finer granularity around those ranges, enabling more precise characterization of subtle spectral features. Conversely, frequency regions that exhibit consistently low power or minimal variation are maintained as coarser partitions, as these regions contribute less discriminative information for neurological inference. This adaptive redistribution ensures that computational resources are dynamically focused on frequency bands that are most relevant to the current neurological state, enhancing both efficiency and analytical sensitivity.

Time-frequency feature extraction within these adaptive frequency partitions is performed by generating multi-dimensional feature representations that capture complementary aspects of neural activity. Temporally aggregated energy values are computed to summarize sustained activation within each partition, while inter-frequency coupling indicators are derived to quantify relationships such as modulation or cross-band interactions that reflect coordinated neural processing. In parallel, inter-channel synchrony measurements are calculated to assess the degree of temporal alignment and coherence between signals from different scalp regions, providing insight into functional connectivity patterns. These features are computed within each adaptive temporal window to preserve temporal context. To ensure stability and comparability across time, the resulting feature representations are normalized using rolling statistical baselines that are derived exclusively from preceding temporal windows that have passed signal quality validation. This normalization compensates for gradual shifts in overall signal amplitude or noise conditions without suppressing genuine neurological changes. The technical effect achieved by this embodiment is a high-resolution yet computationally efficient spectral representation that dynamically adapts to ongoing brain activity, while the technical advancement lies in the joint adaptive partitioning of frequency space and robust normalization strategy that enhances the consistency and discriminative power of the extracted features for downstream neurological inference.

In an embodiment, analyzing the extracted time-frequency features comprises processing the feature representations through multiple inference passes corresponding to different temporal aggregation scales, weighting inference outcomes based on validation confidence associated with each temporal window, and combining the weighted outcomes to form a consolidated neurological inference result for each screening interval, wherein validating signal quality comprises concurrently evaluating signal-to-noise ratio stability, frequency coherence consistency across channels, and temporal continuity across successive adaptive temporal windows, and designating a temporal window as valid only when all evaluated parameters remain within predefined neurological reliability bounds, and wherein inhibiting generation of screening outputs comprises temporarily suspending inference result aggregation when a predefined number of consecutive temporal windows are designated as invalid, and resuming aggregation only after a subsequent sequence of validated temporal windows satisfies a minimum continuity criterion.

In this embodiment, the extracted time-frequency feature representations are analyzed through a multi-scale inference process designed to balance sensitivity to transient neurological events with robustness to short-term signal variability. Each feature set is processed through multiple inference passes that correspond to different temporal aggregation scales, such as short-duration windows that emphasize rapid cognitive changes and longer-duration windows that capture sustained neurological patterns. By evaluating the same underlying features at multiple temporal resolutions, the system is able to distinguish between momentary fluctuations and consistent neurological trends. The inference outcomes generated at each scale are not treated equally; instead, they are weighted according to a validation confidence score associated with the corresponding temporal window. This confidence score reflects the assessed reliability of the signal during that window and directly influences the contribution of each inference pass. The weighted outcomes are then combined to produce a consolidated neurological inference result for each screening interval, thereby reducing susceptibility to noise while maintaining responsiveness to meaningful changes in brain activity. The technical effect of this approach is a more stable and clinically meaningful inference that integrates evidence across time scales rather than relying on a single, potentially noisy observation.

Signal quality validation, which governs the confidence weighting and aggregation logic, is performed concurrently with feature analysis to ensure that only reliable data influences the screening outcome. For each adaptive temporal window, signal-to-noise ratio stability is evaluated to detect excessive noise or amplification artifacts, frequency coherence consistency across channels is assessed to confirm physiologically plausible inter-channel relationships, and temporal continuity across successive windows is examined to identify abrupt discontinuities indicative of artifacts or acquisition faults. A temporal window is designated as valid only when all these parameters remain within predefined neurological reliability bounds that are established during system configuration or clinical calibration. When a predefined number of consecutive temporal windows fail validation, generation of screening outputs is temporarily inhibited by suspending the aggregation of inference results. This prevents unreliable data from producing misleading or clinically unsafe outputs. Aggregation is resumed only after a subsequent sequence of validated temporal windows satisfies a minimum continuity criterion, ensuring that signal quality has been consistently restored. The technical advancement achieved by this embodiment lies in its closed-loop integration of signal validation and inference, which actively governs when and how screening results are produced, thereby enhancing the reliability, safety, and clinical efficacy of real-time neurological screening compared to systems that perform inference without enforcing strict quality-dependent gating.

In an embodiment, updating internal weighting parameters of the trained computational model comprises accumulating feature-outcome associations exclusively from validated temporal windows, computing adjustment magnitudes proportional to longitudinal consistency of screening outcomes, and applying the adjustments incrementally to prevent abrupt shifts in model behavior across successive screening sessions, and wherein storing raw electroencephalographic data and processed outputs comprises organizing stored data into session-indexed records including synchronized raw signal streams, preprocessed signals, transformed representations, extracted feature sets, validation metrics, and inference outcomes, such that each screening session is reconstructable for retrospective clinical assessment.

In this embodiment, the trained computational model is refined over time through a controlled and stability-preserving update mechanism that selectively incorporates reliable evidence from ongoing screening sessions. Feature-outcome associations are accumulated exclusively from temporal windows that have passed signal quality validation, ensuring that only high-integrity data contributes to model adaptation. For each screening session, the system evaluates the longitudinal consistency of the generated screening outcomes across successive validated windows, such that outcomes exhibiting stable and repeatable patterns are assigned higher confidence than those showing sporadic or contradictory behavior. Adjustment magnitudes for the internal weighting parameters of the model are computed in proportion to this longitudinal consistency, thereby favoring gradual reinforcement of consistently observed neurological patterns while suppressing the influence of isolated or transient deviations. These adjustments are applied incrementally across sessions, rather than in a single update step, to prevent abrupt shifts in model behavior that could otherwise compromise interpretability or clinical reliability. The technical effect achieved by this update strategy is a continuously adapting model that remains stable across repeated use, improving personalization and long-term accuracy without introducing drift or overfitting.

In parallel, comprehensive data storage is performed to support traceability, auditability, and retrospective clinical evaluation. Raw electroencephalographic data and all derived processing outputs are organized into session-indexed records that preserve the full processing context of each screening session. Each record includes time-synchronized raw signal streams from all electrodes, preprocessed signals after artifact mitigation and normalization, time-frequency transformed representations, extracted multi-dimensional feature sets, validation metrics associated with each adaptive temporal window, and the resulting inference outcomes. This structured organization ensures that every screening session is fully reconstructable, allowing clinicians or researchers to revisit the original signals, examine intermediate processing stages, and verify or reinterpret the inference results as needed. The technical advancement of this embodiment lies in its integration of reliability-aware model updating with end-to-end data provenance, which not only enhances adaptive screening performance over time but also supports transparency, clinical accountability, and post hoc analysis that are essential for real-world neurological assessment systems.

In an embodiment, generating real-time screening outputs comprises continuously updating a screening likelihood metric by integrating inference results across a sliding sequence of validated adaptive temporal windows, attenuating the influence of transient deviations, and maintaining responsiveness to sustained neurological pattern changes detected during ongoing acquisition, and wherein temporally aligning the signals across the plurality of scalp-mounted electrodes further comprises maintaining a rolling alignment buffer for each digitized signal stream, continuously computing inter-channel phase deviation within overlapping temporal segments, selecting a reference channel exhibiting minimum phase fluctuation relative to the common timing reference, and incrementally correcting phase offsets of remaining channels by applying fractional delay compensation within the rolling alignment buffer prior to preprocessing.

In this embodiment, real-time screening outputs are generated through a continuously updated screening likelihood metric that reflects the evolving neurological state of the subject during ongoing signal acquisition. Rather than producing isolated or instantaneous decisions, the system integrates inference results across a sliding sequence of adaptive temporal windows that have passed signal quality validation. Each new validated inference contributes to the likelihood metric with a controlled weighting, such that transient deviations or short-lived anomalies exert a limited influence, while sustained and consistent neurological patterns progressively dominate the metric. This integration mechanism ensures that the screening output remains stable and clinically meaningful, yet responsive to genuine changes in brain activity as they persist over time. As a result, abrupt fluctuations caused by noise or momentary artifacts are naturally attenuated, whereas enduring deviations associated with neurological conditions or cognitive state changes are promptly reflected in the screening output. The technical effect achieved is a balanced real-time decision signal that combines robustness with sensitivity, which is particularly important for continuous screening applications.

Concurrently, precise temporal alignment across the plurality of scalp-mounted electrodes is maintained through a rolling alignment buffer mechanism that operates prior to preprocessing. For each digitized signal stream, a rolling buffer stores overlapping temporal segments that enable continuous monitoring of inter-channel phase relationships. Within these segments, inter-channel phase deviation is computed to identify relative timing misalignments that may arise from hardware jitter, clock drift, or signal path latency differences. A reference channel is dynamically selected based on exhibiting the minimum phase fluctuation relative to the common timing reference, thereby serving as a stable temporal anchor. Phase offsets of the remaining channels are then incrementally corrected by applying fractional delay compensation within their respective rolling buffers. These corrections are applied gradually to avoid introducing discontinuities or phase artifacts. The technical advancement of this embodiment lies in its ability to preserve fine-grained phase coherence across channels in real time while simultaneously generating stable screening outputs, enabling accurate connectivity analysis and reliable neurological inference that would otherwise be degraded by even small temporal misalignments.

In an embodiment, detecting environmental interference during preprocessing comprises isolating frequency components exhibiting synchronous amplitude modulation across non-adjacent electrodes, correlating the isolated components with temporal patterns of external electromagnetic fluctuation detected across multiple channels, and selectively attenuating the correlated components by dynamically adjusting adaptive filtering coefficients on a per-window basis without altering uncorrelated neurological signal components, and wherein transferring the data blocks into the graphical processing unit memory space comprises allocating separate circular memory buffers for raw preprocessed signal blocks and transformed output blocks, scheduling asynchronous memory copy operations aligned with acquisition timing markers, and enforcing memory access ordering through synchronization primitives such that transformation operations are executed only after complete data block availability is confirmed.

In this embodiment, environmental interference is identified and mitigated during preprocessing through a coordinated spatial-spectral analysis that distinguishes externally induced electromagnetic artifacts from genuine neurological activity. Frequency components within each channel are examined to detect synchronous amplitude modulation patterns that occur simultaneously across non-adjacent electrodes, a characteristic signature of environmental interference such as power-line noise, nearby electronic equipment emissions, or transient electromagnetic disturbances. These components are further correlated with temporal fluctuation patterns observed across multiple channels to confirm their external origin, since true neurological signals typically exhibit localized spatial propagation and physiologically constrained synchrony rather than uniform modulation across distant electrodes. Once correlated components are identified, adaptive filtering coefficients are dynamically adjusted on a per-window basis to selectively attenuate only those frequency components associated with the detected interference. This selective attenuation is performed without altering uncorrelated signal components, thereby preserving the integrity of underlying brain rhythms and avoiding excessive suppression that could distort neurological features. The technical effect achieved is robust rejection of environmental noise under real-world operating conditions, enabling reliable signal acquisition even in electrically noisy environments, while the technical advancement lies in the ability to suppress interference adaptively and selectively rather than applying static notch or broadband filters that indiscriminately remove potentially informative signal content.

In parallel, efficient data handling for high-throughput signal processing is ensured during transfer of data blocks into the graphical processing unit memory space. Separate circular memory buffers are allocated for raw preprocessed signal blocks and for transformed output blocks, allowing continuous streaming without interrupting acquisition. Asynchronous memory copy operations are scheduled in alignment with acquisition timing markers so that data transfer overlaps with computation, minimizing idle time and reducing end-to-end latency. Memory access ordering is strictly enforced using synchronization primitives, ensuring that transformation operations on the graphical processing unit are initiated only after complete data block availability has been confirmed within the corresponding buffer. This guarantees data integrity and temporal correctness of the transformations while sustaining real-time throughput. The technical advancement of this embodiment resides in the tight integration of interference-resilient preprocessing with deterministic, synchronization-safe graphical processing unit data management, enabling stable real-time neurological analysis under both computational and environmental constraints.

Following acquisition, the digitized electroencephalographic signals undergo preprocessing to stabilize baseline drift, suppress motion-related disturbances, and attenuate environmental noise components. This preprocessing stage employs adaptive conditioning logic that continuously estimates signal variance and noise characteristics, allowing filtering parameters to be adjusted in real time rather than relying on static filter configurations. Temporal alignment is further refined by compensating for channel-specific latency differences, ensuring that concurrent neurological events are represented consistently across the entire signal set.

The conditioned electroencephalographic signals are then transmitted to a graphical processing unit configured for high-throughput parallel computation. Within this processing environment, concurrent time-frequency transformations are executed across multiple channels simultaneously. The technique partitions incoming signals into overlapping temporal segments, the lengths of which are dynamically adjusted based on detected signal non-stationarity and rate of spectral change. For signals exhibiting rapid neurological transitions, shorter temporal windows are applied to preserve temporal resolution, while more stable signals are processed using longer windows to enhance frequency resolution. This adaptive segmentation ensures that transient neurological features relevant to Attention Deficit Hyperactivity Disorder are not obscured by rigid analytical constraints.

The time-frequency transformation stage produces multi-dimensional spectral representations of the electroencephalographic signals, capturing both temporal evolution and frequency distribution of brainwave activity. These representations are generated continuously in real time, with the graphical processing unit allocating computation threads across channels and temporal segments to prevent latency accumulation. The technique further refines frequency partitioning by adjusting resolution based on observed spectral density variations, thereby emphasizing neurological frequency ranges that exhibit diagnostically relevant behavior during screening.

From the transformed representations, the technique extracts time-frequency features that characterize neurological activity patterns associated with attentional regulation and cognitive control. Feature extraction includes determining relative energy distributions across frequency ranges, temporal modulation of spectral power, and consistency of frequency relationships over successive temporal windows. These features are selected and updated dynamically, allowing the system to respond to evolving neurological states rather than relying on fixed feature sets.

The extracted features are then processed by a neurological inference procedure that compares current feature patterns with stored neurological reference patterns associated with Attention Deficit Hyperactivity Disorder. This inference procedure is implemented as a trained computational model residing in system memory and accessed by the graphical processing unit during execution. The model evaluates similarity, deviation, and persistence of detected patterns over time, generating an inference output that reflects the likelihood of Attention Deficit Hyperactivity Disorder-related neurological behavior. The inference process operates continuously, refining its assessment as new data becomes available.

To ensure diagnostic reliability, the technique incorporates a validation stage that continuously evaluates signal integrity and inference stability. Validation logic monitors parameters including signal-to-noise characteristics, temporal continuity of detected features, and coherence of frequency behavior across channels. When validation criteria are not satisfied, the technique suppresses or flags screening outputs to prevent unreliable interpretation. This continuous validation ensures that diagnostic indicators are derived only from neurologically meaningful and technically reliable data.

The system further incorporates an adaptive learning mechanism that updates internal inference parameters based on accumulated validated screening outcomes. Over successive screening sessions, the technique adjusts weighting of time-frequency features to better reflect subject-specific and population-level neurological variability. This adaptive refinement improves screening sensitivity and specificity without interrupting real-time operation or requiring offline retraining.

Screening outputs generated by the technique are provided in real time through an output interface that presents neurological indicators, confidence measures, and temporal trends suitable for clinical interpretation. These outputs are updated dynamically as new electroencephalographic data is processed, enabling continuous monitoring rather than single-point assessment. Simultaneously, raw signals, processed features, validation metrics, and screening results are stored in memory for subsequent clinical review and longitudinal analysis.

Computational workload within the graphical processing unit is regulated through an internal workload management procedure that allocates processing resources based on real-time demand. During periods of stable neurological activity, computational intensity is reduced to conserve power, while periods of rapid signal change trigger increased processing allocation to preserve analytical fidelity. This dynamic workload regulation enables prolonged screening sessions without excessive power consumption or thermal stress.

Following acquisition, the digitized electroencephalographic signals undergo preprocessing to stabilize baseline drift, suppress motion-related disturbances, and attenuate environmental noise components. This preprocessing stage employs adaptive conditioning logic that continuously estimates signal variance and noise characteristics, allowing filtering parameters to be adjusted in real time rather than relying on static filter configurations. Temporal alignment is further refined by compensating for channel-specific latency differences, ensuring that concurrent neurological events are represented consistently across the entire signal set.

The conditioned electroencephalographic signals are then transmitted to a graphical processing unit configured for high-throughput parallel computation. Within this processing environment, concurrent time-frequency transformations are executed across multiple channels simultaneously.

The technique partitions incoming signals into overlapping temporal segments, the lengths of which are dynamically adjusted based on detected signal non-stationarity and rate of spectral change. For signals exhibiting rapid neurological transitions, shorter temporal windows are applied to preserve temporal resolution, while more stable signals are processed using longer windows to enhance frequency resolution. This adaptive segmentation ensures that transient neurological features relevant to Attention Deficit Hyperactivity Disorder are not obscured by rigid analytical constraints.

The time-frequency transformation stage produces multi-dimensional spectral representations of the electroencephalographic signals, capturing both temporal evolution and frequency distribution of brainwave activity. These representations are generated continuously in real time, with the

graphical processing unit allocating computation threads across channels and temporal segments to prevent latency accumulation. The technique further refines frequency partitioning by adjusting resolution based on observed spectral density variations, thereby emphasizing neurological frequency ranges that exhibit diagnostically relevant behavior during screening.

From the transformed representations, the technique extracts time-frequency features that characterize neurological activity patterns associated with attentional regulation and cognitive control. Feature extraction includes determining relative energy distributions across frequency ranges, temporal modulation of spectral power, and consistency of frequency relationships over successive temporal windows. These features are selected and updated dynamically, allowing the system to respond to evolving neurological states rather than relying on fixed feature sets.

The extracted features are then processed by a neurological inference procedure that compares current feature patterns with stored neurological reference patterns associated with Attention Deficit Hyperactivity Disorder. This inference procedure is implemented as a trained computational model residing in system memory and accessed by the graphical processing unit during execution. The model evaluates similarity, deviation, and persistence of detected patterns over time, generating an inference output that reflects the likelihood of Attention Deficit Hyperactivity Disorder-related neurological behavior. The inference process operates continuously, refining its assessment as new data becomes available.

To ensure diagnostic reliability, the technique incorporates a validation stage that continuously evaluates signal integrity and inference stability. Validation logic monitors parameters including signal-to-noise characteristics, temporal continuity of detected features, and coherence of frequency behavior across channels. When validation criteria are not satisfied, the technique suppresses or flags screening outputs to prevent unreliable interpretation. This continuous validation ensures that diagnostic indicators are derived only from neurologically meaningful and technically reliable data.

The system further incorporates an adaptive learning mechanism that updates internal inference parameters based on accumulated validated screening outcomes. Over successive screening sessions, the technique adjusts weighting of time-frequency features to better reflect subject-specific

and population-level neurological variability. This adaptive refinement improves screening sensitivity and specificity without interrupting real-time operation or requiring offline retraining.

Screening outputs generated by the technique are provided in real time through an output interface that presents neurological indicators, confidence measures, and temporal trends suitable for clinical interpretation. These outputs are updated dynamically as new electroencephalographic data is processed, enabling continuous monitoring rather than single-point assessment. Simultaneously, raw signals, processed features, validation metrics, and screening results are stored in memory for subsequent clinical review and longitudinal analysis.

Computational workload within the graphical processing unit is regulated through an internal workload management procedure that allocates processing resources based on real-time demand. During periods of stable neurological activity, computational intensity is reduced to conserve power, while periods of rapid signal change trigger increased processing allocation to preserve analytical fidelity. This dynamic workload regulation enables prolonged screening sessions without excessive power consumption or thermal stress.

The system and method for real-time ADHD screening disclosed herein are implemented as a dedicated computing machine configured to receive raw EEG signals from multiple electrodes positioned on a subject's scalp. The EEG acquisition structure comprises signal amplifiers, analog-to-digital converters, and synchronization circuits that ensure temporal alignment across channels. The digitized EEG signals are transmitted to a processing bus coupled directly to a GPU-based computational unit.

The GPU processing unit is structurally integrated within the machine housing and is configured to execute parallel time-frequency transformations on incoming EEG data streams. Unlike traditional CPU-based systems, the GPU executes concurrent spectral decomposition operations using adaptive windowing and variable resolution kernels, allowing real-time extraction of transient frequency components associated with ADHD-related neurological activity. This enables continuous monitoring without buffering delays or offline computation.

The processed EEG data is forwarded to a time-frequency inference processor implemented as a logical layer within the GPU computation pipeline. This processor dynamically adjusts frequency band segmentation based on detected signal characteristics and neurological variability, enabling precise identification of theta, beta, and related spectral patterns relevant to ADHD screening. Computational validation routines continuously assess signal integrity, noise thresholds, and temporal consistency to ensure reliability of the inferred features.

A neurological inference unit, implemented using machine learning models stored within the machine's memory subsystem, receives validated spectral features and performs pattern recognition to determine ADHD likelihood. The inference unit adapts its internal parameters over time based on accumulated screening data, enabling progressive improvement in screening accuracy. The output of the inference process is generated in real time and communicated to a clinical interface unit.

The clinical interface unit comprises display hardware, alert signaling components, and data export interfaces that allow clinicians to visualize screening outcomes, monitor neurological trends, and store diagnostic records. The entire machine structure is enclosed within a medically compliant housing that supports electromagnetic shielding, thermal regulation, and secure data access. Energy-efficient power management circuits regulate GPU workload to ensure sustained operation during prolonged screening sessions.

The disclosed system thereby provides a structurally integrated, machine-based solution capable of performing real-time ADHD screening through GPU-accelerated EEG time-frequency inference, overcoming the computational and diagnostic limitations of existing systems while maintaining clinical reliability and scalability.

The invention is applicable in hospitals, neurological clinics, diagnostic centers, research institutions, and telemedicine environments requiring real-time ADHD screening. The system may be deployed as a standalone diagnostic machine or integrated with existing EEG infrastructure to enhance screening efficiency and accuracy.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A method for real-time screening of Attention Deficit Hyperactivity Disorder using electroencephalographic signals, comprising the steps of:
acquiring analog electroencephalographic signals from a plurality of scalp-mounted electrodes and converting the acquired signals into synchronized digitized signal streams;
preprocessing the digitized signal streams by stabilizing baseline drift, attenuating artifacts, and temporally aligning signals across multiple channels;
executing, using a graphical processing unit configured for parallel computation, concurrent time-frequency transformations on the preprocessed signal streams in real time;
dynamically segmenting the transformed signals into adaptive temporal windows and frequency partitions based on detected signal characteristics;
extracting time-frequency features representative of neurological activity from the segmented signals;
analyzing the extracted time-frequency features using a neurological inference process to determine patterns associated with Attention Deficit Hyperactivity Disorder;
validating signal quality, temporal consistency, and frequency stability prior to generating screening results; and
generating real-time screening outputs indicative of a likelihood of Attention Deficit Hyperactivity Disorder, wherein acquiring the electroencephalographic signals comprises amplifying low-amplitude brainwave signals, digitizing the amplified signals using analog-to-digital conversion, and synchronizing the digitized signals across all channels using a common timing reference, wherein preprocessing the digitized signal streams comprises detecting motion-related disturbances, electrode impedance variations, and environmental interference, and selectively suppressing the detected disturbances using adaptive filtering parameters derived from real-time signal variance measurements, wherein executing concurrent time-frequency transformations comprises distributing signal processing tasks across multiple parallel computation threads within the graphical processing unit such that multiple electroencephalographic channels are processed simultaneously without introducing acquisition-to-inference latency, wherein dynamically segmenting the transformed signals comprises adjusting temporal window lengths in response to detected non-stationary signal behavior and modifying frequency resolution based on observed spectral transitions associated with neurological activity.

2. The method of claim 1, wherein extracting the time-frequency features comprises identifying frequency band energy distributions, temporal power variations, and cross-band relationships indicative of attention-related neurological patterns, wherein analyzing the extracted time-frequency features comprises comparing the extracted features with stored neurological reference patterns using a trained computational model to determine similarity to Attention Deficit Hyperactivity Disorder-associated signatures; and wherein validating signal quality comprises continuously monitoring signal-to-noise characteristics, frequency coherence, and temporal continuity, and inhibiting generation of screening outputs when one or more validation parameters fall outside predefined neurological reliability limits.

3. The method of claim 1, further comprising:
updating internal weighting parameters of the trained computational model based on accumulated screening outcomes and validated signal features to improve screening accuracy over successive screening sessions; and
storing raw electroencephalographic data, processed time-frequency features, validation metrics, and screening results in a memory unit for subsequent clinical review and longitudinal neurological assessment.

4. The method of claim 1, wherein amplifying the low-amplitude brainwave signals comprises applying channel-specific gain control by initially measuring baseline amplitude distributions for each of the plurality of scalp-mounted electrodes during an initialization interval, dynamically adjusting amplification factors for each channel based on the measured distributions to prevent saturation and clipping, and continuously recalibrating the amplification factors during acquisition in response to slow amplitude drift detected in the digitized signal streams, wherein synchronizing the digitized signals across all channels using the common timing reference comprises embedding timing markers into each digitized signal stream at acquisition, detecting timing deviations by comparing marker alignment across channels, computing channel-specific timing offsets at sub-sample resolution, and correcting the offsets by interpolative resampling prior to preprocessing to maintain phase coherence across the plurality of scalp-mounted electrodes.

5. The method of claim 1, wherein detecting motion-related disturbances comprises monitoring abrupt amplitude excursions, transient broadband spectral energy increases, and inter-channel decorrelation events occurring within predefined temporal proximity, classifying detected events as motion-induced disturbances when the events simultaneously affect a minimum subset of spatially adjacent electrodes, and tagging corresponding signal segments for selective suppression during preprocessing; and wherein selectively suppressing the detected disturbances comprises calculating adaptive filtering parameters by estimating real-time signal variance, spectral flatness, and temporal persistence within the tagged signal segments, applying attenuation selectively to affected frequency components while preserving uncorrupted frequency components, and reintegrating the filtered segments into the digitized signal streams without introducing discontinuities between adjacent unfiltered segments.

6. The method of claim 1, wherein detecting electrode impedance variations comprises continuously estimating impedance-related signal distortions by evaluating low-frequency drift magnitude, noise floor elevation, and phase instability for each channel, comparing the estimated distortions against baseline impedance profiles obtained during initialization, and dynamically modifying preprocessing parameters to reduce the influence of channels exhibiting impedance instability on subsequent processing stages, and wherein executing concurrent time-frequency transformations using the graphical processing unit comprises partitioning each preprocessed signal stream into overlapping data blocks, transferring the data blocks into a graphical processing unit memory space using asynchronous data transfer operations, executing parallel transformation operations on the data blocks across multiple processing cores, and assembling transformed outputs in a time-ordered sequence to preserve temporal continuity; and wherein distributing signal processing tasks across multiple parallel computation threads comprises mapping individual electroencephalographic channels to independent thread groups, assigning transformation workloads based on channel-specific signal complexity determined from variance and spectral density measurements, and dynamically balancing thread execution to prevent processing delays in channels exhibiting higher computational demand.

7. The method of claim 1, wherein dynamically segmenting the transformed signals into adaptive temporal windows comprises continuously evaluating non-stationary behavior by tracking short-term energy fluctuations and spectral redistribution within the transformed signals, decreasing temporal window duration upon detection of rapid transitions indicative of cognitive state changes, and increasing temporal window duration during periods of sustained spectral stability to capture extended neurological patterns, and wherein dynamically segmenting the transformed signals further comprises recalculating window overlap ratios in response to detected transition frequency, increasing overlap during high-transition intervals to improve temporal resolution and reducing overlap during stable intervals to minimize redundant computation.

8. The method of claim 7, wherein decreasing and increasing the temporal window duration further comprises computing a transition intensity score based on combined temporal energy gradient and frequency redistribution rate within each transformed signal segment, mapping the transition intensity score to a bounded temporal window adjustment range, and applying window duration changes gradually across successive segments to prevent abrupt discontinuities in feature extraction, and wherein generating the multi-dimensional feature representations further comprises computing channel-pair interaction descriptors by measuring time-lagged co-activation patterns within each adaptive temporal window, weighting the interaction descriptors based on channel validation confidence derived during signal quality validation, and integrating the weighted interaction descriptors with channel-wise features to form a composite feature set used for neurological inference.

9. The method of claim 1, wherein dynamically segmenting the transformed signals into frequency partitions comprises monitoring relative power shifts across frequency ranges, reallocating frequency partition boundaries toward ranges exhibiting increased neurological relevance, and maintaining coarser partitions in ranges exhibiting low informational contribution, such that computational emphasis is adaptively concentrated on neurologically active frequency regions, and wherein extracting the time-frequency features comprises generating multi-dimensional feature representations by combining temporally aggregated energy values, inter-frequency coupling indicators, and inter-channel synchrony measurements computed within each adaptive temporal window, and normalizing the feature representations using rolling statistical baselines derived from preceding validated windows.

10. The method of claim 2, wherein analyzing the extracted time-frequency features comprises processing the feature representations through multiple inference passes corresponding to different temporal aggregation scales, weighting inference outcomes based on validation confidence associated with each temporal window, and combining the weighted outcomes to form a consolidated neurological inference result for each screening interval, wherein validating signal quality comprises concurrently evaluating signal-to-noise ratio stability, frequency coherence consistency across channels, and temporal continuity across successive adaptive temporal windows, and designating a temporal window as valid only when all evaluated parameters remain within predefined neurological reliability bounds, and wherein inhibiting generation of screening outputs comprises temporarily suspending inference result aggregation when a predefined number of consecutive temporal windows are designated as invalid, and resuming aggregation only after a subsequent sequence of validated temporal windows satisfies a minimum continuity criterion.

11. The method of claim 3, wherein updating internal weighting parameters of the trained computational model comprises accumulating feature-outcome associations exclusively from validated temporal windows, computing adjustment magnitudes proportional to longitudinal consistency of screening outcomes, and applying the adjustments incrementally to prevent abrupt shifts in model behavior across successive screening sessions, and wherein storing raw electroencephalographic data and processed outputs comprises organizing stored data into session-indexed records including synchronized raw signal streams, preprocessed signals, transformed representations, extracted feature sets, validation metrics, and inference outcomes, such that each screening session is reconstructable for retrospective clinical assessment.

12. The method of claim 1, wherein generating real-time screening outputs comprises continuously updating a screening likelihood metric by integrating inference results across a sliding sequence of validated adaptive temporal windows, attenuating the influence of transient deviations, and maintaining responsiveness to sustained neurological pattern changes detected during ongoing acquisition, and wherein temporally aligning the signals across the plurality of scalp-mounted electrodes further comprises maintaining a rolling alignment buffer for each digitized signal stream, continuously computing inter-channel phase deviation within overlapping temporal segments, selecting a reference channel exhibiting minimum phase fluctuation relative to the common timing reference, and incrementally correcting phase offsets of remaining channels by applying fractional delay compensation within the rolling alignment buffer prior to preprocessing.

13. The method of claim 1, wherein detecting environmental interference during preprocessing comprises isolating frequency components exhibiting synchronous amplitude modulation across non-adjacent electrodes, correlating the isolated components with temporal patterns of external electromagnetic fluctuation detected across multiple channels, and selectively attenuating the correlated components by dynamically adjusting adaptive filtering coefficients on a per-window basis without altering uncorrelated neurological signal components, and wherein transferring the data blocks into the graphical processing unit memory space comprises allocating separate circular memory buffers for raw preprocessed signal blocks and transformed output blocks, scheduling asynchronous memory copy operations aligned with acquisition timing markers, and enforcing memory access ordering through synchronization primitives such that transformation operations are executed only after complete data block availability is confirmed.

14. A system for real-time screening of Attention Deficit Hyperactivity Disorder implementing the method of claim 1, comprising:
an electroencephalographic signal acquisition unit configured to receive analog brainwave signals from a plurality of scalp-mounted electrodes and convert the received signals into digitized signal streams;
a preprocessing unit operatively coupled to the electroencephalographic signal acquisition unit and configured to condition the digitized signal streams by performing baseline stabilization, artifact attenuation, and temporal alignment across multiple channels;
a graphical processing unit operatively coupled to the preprocessing unit, the graphical processing unit being configured as a parallel computation processor to execute concurrent time-frequency transformations on the conditioned signal streams in real time;
a time-frequency inference processor implemented within the graphical processing unit and configured to dynamically segment the conditioned signal streams into adaptive temporal windows and frequency partitions based on detected signal characteristics;
a neurological inference unit operatively coupled to the graphical processing unit and configured to analyze extracted time-frequency features to determine neurological patterns associated with Attention Deficit Hyperactivity Disorder;
a validation unit configured to continuously assess signal integrity, frequency stability, and temporal consistency of the processed signals prior to generation of screening outputs; and
an output interface unit configured to generate real-time screening indicators indicative of a likelihood of Attention Deficit Hyperactivity Disorder for clinical review.

15. The system of claim 14, wherein the electroencephalographic signal acquisition unit comprises a plurality of amplification circuits, analog-to-digital conversion circuits, and synchronization circuits configured to maintain channel-level temporal coherence across all digitized signal streams prior to preprocessing, wherein the preprocessing unit is configured to detect and suppress motion-induced artifacts, electrode impedance fluctuations, and environmental noise components by applying adaptive filtering parameters derived from real-time signal variance measurements; and wherein the graphical processing unit is configured to execute multiple time-frequency transformations concurrently across the plurality of electroencephalographic channels by distributing computation threads such that latency between signal acquisition and feature extraction remains within a predetermined real-time threshold.

16. The system of claim 14, wherein the time-frequency inference processor is configured to vary temporal window lengths and frequency resolution in response to detected changes in signal non-stationarity, cognitive state transitions, or abrupt spectral deviations associated with neurological activity; wherein the neurological inference unit comprises a trained computational model stored in a memory unit, the computational model being configured to correlate extracted time-frequency features with stored neurological reference patterns indicative of Attention Deficit Hyperactivity Disorder; and wherein the neurological inference unit is further configured to update internal weighting parameters of the computational model based on accumulated screening outcomes and validated signal patterns, thereby enabling adaptive improvement of screening accuracy over successive screening sessions.

17. The system of claim 14, wherein the validation unit is configured to inhibit generation of screening outputs when one or more signal quality parameters fall outside predefined neurological reliability limits, thereby preventing screening decisions based on degraded or unreliable electroencephalographic data; wherein the output interface unit is configured to provide continuous visualization of neurological trends, screening confidence indicators, and temporal progression of inferred attention-related patterns through a clinician-accessible display interface, and wherein said system comprising a memory unit operatively coupled to the preprocessing unit, graphical processing unit, and neurological inference unit, the memory unit being configured to store raw electroencephalographic data, processed time-frequency features, validation metrics, and screening results for subsequent clinical analysis.
